(19) United States
(12) Patent Application Publication
(10) Pub. No.: US 2023/0028744 A1
(43) Pub. Date: Jan. 26, 2023

(54) NANOSTRUCTURE COMPRISING MAGNETIC NANOPARTICLES AND TRANSFERRIN FAMILY PROTEIN, METHOD FOR PREPARING THE SAME, AND METHOD FOR ISOLATING OR CONCENTRATING EXTRACELLULAR VESICLES OR PATHOGEN

(71) Applicant: UIF (University Industry Foundation), Yonsei University, Seoul (KR)

(72) Inventors: Yong SHIN, Seoul (KR); Thuy Nguyen Thi Dao, Seoul (KR)

(73) Assignee: UIF (University Industry Foundation), Yonsei University, Seoul (KR)

(21) Appl. No.: 17/860,537

(22) Filed: Jul. 8, 2022

(30) Foreign Application Priority Data
Jul. 16, 2021 (KR) 10-2021-0093618

(51) Int. Cl. G01N 1/40; C07K 14/47; C12N 1/02; C12N 1/20

(52) U.S. Cl. CPC G01N 1/405; C07K 14/473; C12N 1/02; C12N 1/20

(57) ABSTRACT

A nanostructure for isolating or concentrating extracellular vesicles or a pathogen, includes a transferrin family protein linked on magnetic nanoparticles. The nanostructure includes a transferrin family protein, and thus has selectivity for a pathogen or extracellular vesicles capable of binding to the transferrin family protein, and the synthesized nanostructure is positively (+) charged. The nanostructure includes magnetic nanoparticles, a target material is easily and simply isolated from other materials by magnetism when a magnetic field is applied.

### CROSS-REFERENCE TO RELATED APPLICATION

This application claims priority to and the benefit of Korean Patent Application No. 10-2021-0093618, filed on Jul. 16, 2021, the disclosure of which is incorporated herein by reference in its entirety.

### BACKGROUND

The present invention relates to a nanostructure including magnetic nanoparticles and a transferrin family protein, a method for preparing the same, and a

Since extracellular vesicles (EVs) secreted from cells, particularly exosomes and microvesicles, play a role in transferring information between in vivo cells and include a large amount of biomarkers (genes, nucleic acids, proteins, and the like), research is being actively conducted on the use of extracellular vesicles for diagnosis of various diseases such as cancer or as a drug delivery vehicle or therapeutic agent. Recently, the range of using extracellular vesicles has expanded to cosmetics or health foods.

However, since extracellular vesicles have a small size and a low density and are difficult to obtain, there is a disadvantage in that a complicated experimental method using an expensive antibody and expensive elaborate equipment such as an ultracentrifuge are required in order to selectively isolate or screen only the extracellular vesicles.

Therefore, there is a need for a method capable of easily isolating or concentrating extracellular vesicles at a reasonable cost.

### SUMMARY

An object of the present invention is to provide a material capable of being used for simple and easy isolation of extracellular vesicles.

Another object of the present invention is to provide a method for isolating extracellular vesicles using the material.

Still another object of the present invention is to provide a method for preparing the material.

The present inventors found that using a transferrin family protein capable of binding to extracellular vesicles and magnetic nanoparticles, and a positive (+) charge of a structure, extracellular vesicles can be isolated by binding to the transferrin family protein, the magnetic force from a magnetic field, and the attractive force between the positive charge of the structure and the negative surface charge of the extracellular vesicles, thereby completing the present invention. Further, the present inventors found that the transferrin family protein can also be applied to the isolation of a pathogen that can be bound, and can also be used to concentrate such a target material.

Therefore, according to an aspect of the present invention, provided is a nanostructure for isolating or concentrating extracellular vesicles or a pathogen, including a transferrin family protein linked on magnetic nanoparticles.

According to another aspect of the present invention, provided is a method for isolating or concentrating extracellular vesicles or a pathogen using the nanostructure.

According to still another aspect of the present invention, provided is a method for preparing a nanostructure for concentrating or isolating a pathogen or extracellular vesicles, the method including: (i) a step of treating magnetic nanoparticles with a silane coupling agent; (ii) a step of treating the product obtained in Step (i) with a polyfunctional crosslinking agent; (iii) a step of converting a polyfunctional end group of the product obtained in Step (ii) to a thiol group; (iv) a step of treating the product obtained in Step (iii) with a maleimide-based crosslinking agent; and (v) a step of treating the product obtained in Step (iv) with a transferrin family protein.

Since the nanostructure according to the present invention includes a transferrin family protein, and thus has selectivity for a pathogen or extracellular vesicles capable of binding to the transferrin family protein, and the synthesized nanostructure is positively (+) charged, the nanostructure may bind to pathogens and cells whose surfaces are negatively (−) charged, and thus pathogens and extracellular vesicles may be isolated or concentrated with excellent efficiency because various methods work together as described above. Furthermore, since the nanostructure includes magnetic nanoparticles, a target material can be easily and simply isolated by magnetic force when a magnetic field is applied. Therefore, the pathogen or extracellular vesicles can be concentrated or isolated inexpensively, simply and easily, with excellent efficiency in a short period of time, without using an expensive antibody or requiring a complicated experimental procedure and elaborate equipment.

### BRIEF DESCRIPTION OF THE DRAWINGS

FIG. 1 is a view exemplifying a method for isolating or concentrating extracellular vesicles or a pathogen according to an exemplary embodiment of the present invention;

FIG. 2 is a view schematically illustrating a method for preparing a nanostructure according to an exemplary embodiment of the present invention;

FIGS. 3A and 3B are SEM images showing the uniform particle size distribution and shape of pure Fe₃O₄ magnetic nanoparticles (FIG. 3A) and a structure (FIG. 3B) prepared in the Synthesis Example of the Example according to an exemplary embodiment of the present invention;

FIGS. 4A and 4B illustrate, as a graph, a size distribution plot analyzed by ImageJ by randomly measuring the sizes of pure Fe₃O₄ magnetic nanoparticles (FIG. 4A) and 200 particles of a structure (FIG. 4B) prepared in the Synthesis Example of the Example according to an exemplary embodiment of the present invention;

FIG. 5 illustrates the zeta potentials of commercially available Fe₃O₄ MNPs and synthesized Fe₃O₄ MNP, bisMPA-MNP, and LF-bis-MPA-MNP;

FIG. 6A is a view illustrating FT-IR spectra, and FIG. 6B is a UV-visible light spectroscopy spectrum;

FIG. 7A is a graph showing the experimental results for optimizing the LF concentration for coating onto MNPs, and FIG. 7B illustrates the experimental results for optimizing the LF-bis-MPA-MNP concentration;

FIGS. 8A and 8B illustrate the results of concentrating and detecting Salmonella (Gram-negative bacterium) (FIG. 8A) and Brucella (FIG. 8B) in PBS samples using the structure of the present invention at various dilutions;

FIGS. 9A to 9E are graphs showing the results of isolating extracellular vesicles using ultracentrifugation (UC), a total exosome isolation (TEI) kit, a dimethyl suberimidate/thin film sample processing (DTS) chip, and LF-bis-MPA-MNP and analyzing the concentration according to the size thereof;

FIGS. 10A and 10B are the results of immunoblotting EVs isolated by UC, TEI, and LF-bis-MPA-MNP; and

FIGS. 11A and 11B show the results of comparing Ct values as a graph after qRT-PCR is performed on exosomes miR-21, miR-31, and U6 released from an HCT116 cell line.

### DETAILED DESCRIPTION

Hereinafter, the present invention will be described in detail.

The terms used in the present application are used only to describe specific embodiments, and are not intended to limit the present invention. Unless defined otherwise, all terms used herein, including technical or scientific terms, have the same meaning as commonly understood by a person with ordinary skill in the art to which the present invention pertains.

Throughout the specification, when a part “includes”, “contains” and “has” a constituent element, it means that other constituent elements may be further included unless otherwise specifically defined.

According to an aspect of the present invention, provided is a nanostructure for isolating or concentrating extracellular vesicles or a pathogen, including a transferrin family protein linked on magnetic nanoparticles.

The magnetic nanoparticles are not particularly limited as long as they are nano-sized particles that can react to a magnetic field and can be bonded to an organic material by surface modification, and may be, for example, iron oxide (Fe₃O₄) nanoparticles. The magnetic nanoparticles may have a diameter in a range of 10 to 120 nm, and may have a diameter in a range of, for example 40 to 70 nm.

The transferrin family protein may be at least one selected from the group consisting of lactoferrin, serotransferrin, ovotransferrin, milk transferrin, and melanotransferrin), and may be, for example, lactoferrin. These transferrin family proteins may be obtained by synthesis or extraction, and include those derived from humans or non-human animals. In addition, the lactoferrin may be selected from the group consisting of hololactoferrin, apolactoferrin and asialolactoferrin.

As used herein, the term “extracellular vesicles” includes all nano-sized (30 to 2,000 nm) extracellularly released vesicles consisting of a phospholipid bilayer, which is the same component as the structure of the cell membrane. Therefore, the term includes “exosomes” and “microvesicles” which are released from cells. Furthermore, the term “extracellular vesicles” is used to mean including ectosomes, microparticles, tolerosomes, prostatosomes, cardiosomes and vexosomes.

The pathogen is not particularly limited as long as it can bind to a transferrin family protein such as lactoferrin, and may be a virus, a bacterium, or a fungus. The bacteria may be Gram-negative bacteria, for example, Escherichia coli, Salmonella, Shigella, Typhus, Vibrio cholerae, Neisseria gonorrhoeae, Neisseria meningitidis, and the like.

The nanostructure may have a size in a range of 20 to 150 nm, and for example, may have a size in a range of 40 to 100 nm. Further, the nanostructure may exhibit a positive surface charge. The nanostructure as described above may exhibit a positive surface charge to attract pathogens such as viruses, bacteria, and fungi or extracellular vesicles, and accordingly, various methods such as binding of extracellular vesicles or pathogens by a transferrin family protein and magnetic force by magnetic nanoparticles work together to enable isolation or concentration of extracellular vesicles or a pathogen with high efficiency in a short period of time.

The nano structure may further include a linking portion between the magnetic nanoparticles and the transferrin family protein. Examples of the linking portion include a polyfunctional crosslinking portion or a linking portion derived from a silane coupling agent. Therefore, according to an exemplary embodiment of the present invention, a nanostructure including a polyfunctional crosslinking portion and/or a linking portion derived from a silane coupling agent may be provided. According to another exemplary embodiment of the present invention, a nanostructure including: magnetic nanoparticles; a linking portion derived from a silane coupling agent on the magnetic nanoparticles; a polyfunctional crosslinking portion on the linking portion derived from the silane coupling agent; and a transferrin family protein bound to the polyfunctional crosslinking portion may be provided.

The polyfunctional crosslinking portion may be derived from a carboxylic acid having an alcohol-based polyfunctional portion, for example, a carboxylic acid having two or more hydroxy groups. Examples of the carboxylic acid having an alcohol-based polyfunctional portion include 2,2-bis (hydroxymethyl) propionic acid (also referred to as bis-MPA), 2,2-bis(hydroxymethyl) butyric acid, but are not limited thereto. When the nanostructure of the present invention has such a polyfunctional crosslinking portion, the nanostructure may have the shape of a dendrimer. Therefore, according to an exemplary embodiment of the present invention, a nanostructure having the shape of a dendrimer may be provided.

Further, the silane coupling agent has two or more different reactive groups in the molecule, one of which is a reactive group that chemically binds to an inorganic material, and the other is a reactive group that chemically binds to an organic material. Therefore, in the nanostructure according to the present invention, the silane coupling agent binds to magnetic nanoparticles through a chemical reaction to form a linking portion, thereby allowing the inorganic magnetic nanoparticles to be chemically linked to other linking portions or transferrin family proteins. Examples of the silane coupling agent typically include vinyl-based, epoxy-based, styryl-based, methacryl-based, acryl-based, amino-based, ureido-based, isocyanurate-based, mercapto-based silane coupling agents, and the like. According to an exemplary embodiment of the present invention, the silane coupling agent coated on the magnetic nanoparticles is an amino-based silane coupling agent. For example, the silane coupling agent of the present invention may be an amino-based silane coupling agent selected from 3-aminopropyltriethoxysilane (APTES) and 3-aminopropylmethoxysilane (APTMS), but is not limited thereto.

According to an exemplary embodiment of the present invention, a nanostructure represented by the following Chemical Formula 1 may be provided.

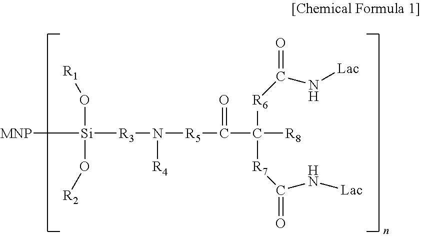

In Chemical Formula 1,

MNP represents a magnetic nanoparticle,

Lac represents a transferrin family protein,

R₁ and R₂ are each independently an alkyl group having 1 to 5 carbon atoms,

R₃, R₅, R₆, and R₇ are each independently an alkylene group having 1 to 5 carbon atoms,

R₄ is a hydrogen atom or an alkyl group having 1 to 5 carbon atoms, and

n is an integer greater than or equal to 1.

Here, the “alkyl (or alkylene) group having 1 to 5 carbon atoms” may be a straight-chained or branched alkyl (or alkylene) group, and includes, for example, methyl (or methylene), ethyl (or ethylene), propyl (or propylene), isopropyl (or isopropylene), n-butyl (or n-butylene), isobutyl (or isobutylene), t-butyl (or t-butylene), and the like.

According to another aspect of the present invention, provided is a method for isolating or concentrating extracellular vesicles or a pathogen using the above-described nanostructure.

According to an exemplary embodiment of the present invention, the method for isolating or concentrating extracellular vesicles or a pathogen may include (a) a step of bringing the nanostructure according to the present invention into contact with extracellular vesicles or a pathogen and (b) a step of applying a magnetic field.

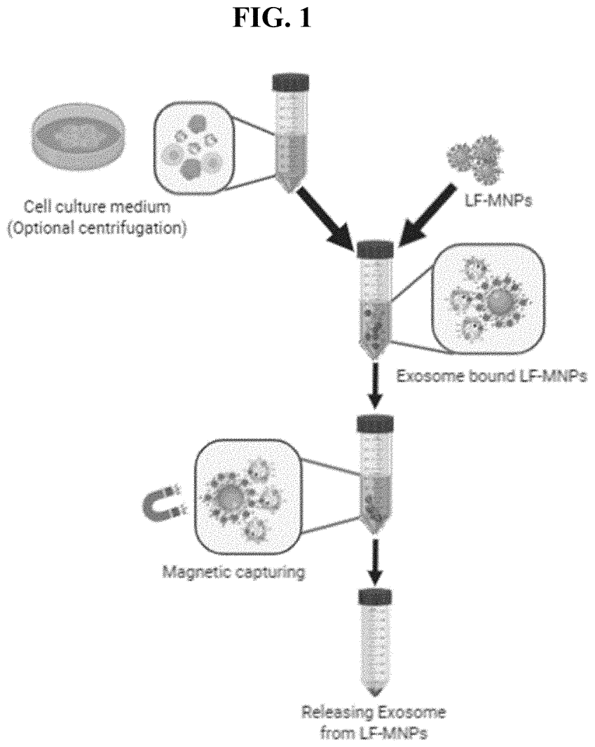

Further, according to another exemplary embodiment of the present invention, referring to FIG. 1, the method for isolating or concentrating extracellular vesicles or a pathogen may include: (a) a step of bringing the nanostructure according to the present invention into contact with extracellular vesicles or a pathogen; (b) a step of applying a magnetic field; (c) a step of isolating a material captured by the magnetic force from the magnetic field; and (d) a step of isolating the extracellular vesicles or the pathogen from the structure. Here, the magnetic field may be applied, for example, by bringing a magnet into contact with a container including target extracellular vesicles or a target pathogen. In addition, Step (c) may be performed, for example, by discharging the material, other than the target material remaining in the container by the magnet, from the container. Step (d) may be performed, for example, by performing a treatment of cutting a linking portion on the nanostructure.

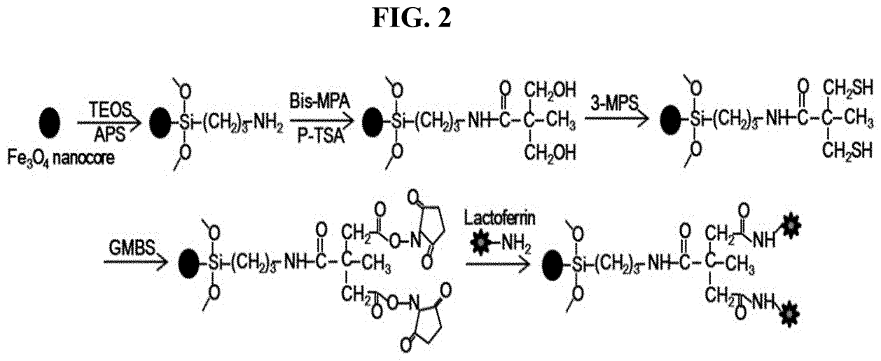

Furthermore, according to still another aspect of the present invention, according to FIG. 2, provided is a method for preparing a nanostructure for isolating or concentrating extracellular vesicles or a pathogen, the method including: (i) a step of treating magnetic nanoparticles with a silane coupling agent; (ii) a step of treating the product obtained in Step (i) with a polyfunctional crosslinking agent; (iii) a step of converting a polyfunctional end group of the product obtained in Step (ii) to a thiol group; (iv) a step of treating the product obtained in Step (iii) with a maleimide-based crosslinking agent; and (v) a step of treating the product obtained in Step (iv) with a transferrin family protein.

In the method for preparing a nanostructure, the silane coupling agent in Step (i) and the polyfunctional crosslinking agent in Step (ii) are as described above. Step (iii) may be performed by adding any material capable of converting a hydroxyl group to a thiol group, and examples of the material include 3-mercaptopropyl trimethoxysilane. Examples of the maleimide-based crosslinking agent in Step (iv) include N-(γ-maleimidobutyryloxy)succinimide ester (GMBS), ε-maleimidocaproic acid N-hydroxysuccinimide ester (EMCS), κ-maleimidoundecanoic acid N-succinimidyl ester (KMUA), m-maleimidobenzoyl-N-hydroxysuccinimide ester (MBS), and the like, but are not limited thereto.

Hereinafter, the present invention will be described in more detail with reference to exemplary embodiments of the present invention. Since the exemplary embodiments are presented for the purpose of describing the present invention, the present is not limited thereto.

### [Synthesis Example] Synthesis of Structure According to Present Invention

(1) Synthesis of Fe₃O₄ Nanoparticles

Approximately 23.5 g of FeCl₃.6H₂O and 8.6 g of FeSO₄.7H₂O (molar ratio of 1:2) were dissolved in 600 ml of distilled water, and 34 ml of NH₄OH was added thereto at approximately 600 rpm and 85° C. for 1 hour. pH was adjusted from 9 to 14. A black precipitate of Fe₃O₄ was produced. After the precipitate was collected with a magnet, the precipitate was washed several times with distilled water until the pH of the solution became neutral.

(2) Modification of Fe₃O₄ Nanoparticles Using Bis-MPA

Magnetic nanoparticles (MNPs) coated with a monofunctional bis-MPA dendrimer were synthesized using an inorganic sol-gel reaction and polycondensation.

Briefly, Fe₃O₄ nanoparticles, hexane (or toluene), Igepal (registered trademark) CO-520 and an ammonia solution (25%) were stirred at 1,000 rpm for 30 minutes. Tetraethyl orthosilicate (TEOS, 98%) was added dropwise to the reaction mixture, and the resulting mixture was stirred overnight. Next, 3-aminopropyltriethoxysilane (APTES, 99%) was added dropwise to the mixture, and the resulting mixture was stirred overnight. Fe₃O₄—NH₂ nanoparticles were washed with methanol and dried in a vacuum desiccator for 1 hour. Fe₃O₄—NH₂, 2,2-bis(hydroxymethyl)propionic acid (bis-MPA), and p-toluenesulfonic acid monohydrate (p-TSA) were mixed, and the resulting mixture was stirred at 200° C. for 1 hour. The mixture was cooled to room temperature and dispersed by ultrasonication in methanol for 30 minutes. The MNPs were centrifuged, washed and re-dispersed in methanol.

(3) Immobilization of Bis-MPA Fe₃O₄ Using Lactoferrin

Lactoferrin was immobilized on the MNP by attaching bis-MPA Fe₃O₄ to a thiol-modified surface through a heterobifunctional crosslinking agent N-(γ-maleimidobutyryloxy) succinimide ester (GMBS).

After the hydroxyl group on the surface of the MNPs was converted to a thiol functional group using a 4% (v/v) 3-mercaptopropyl trimethoxysilane (3-MPS) at room temperature, the MNPs were washed with ethanol and cultured at room temperature for 1 hour using a 1 mM N-(γ-maleimidobutyryloxy)succinimide ester (GMBS). Lactoferrin was reacted on the GMBS-immobilized surface at room temperature for 1 hour. The modified MNPs were used immediately or stored at 4° C.

### [Evaluation Example 1] Preparation Confirmation and Characteristic Evaluation of Structure

(1) SEM Image

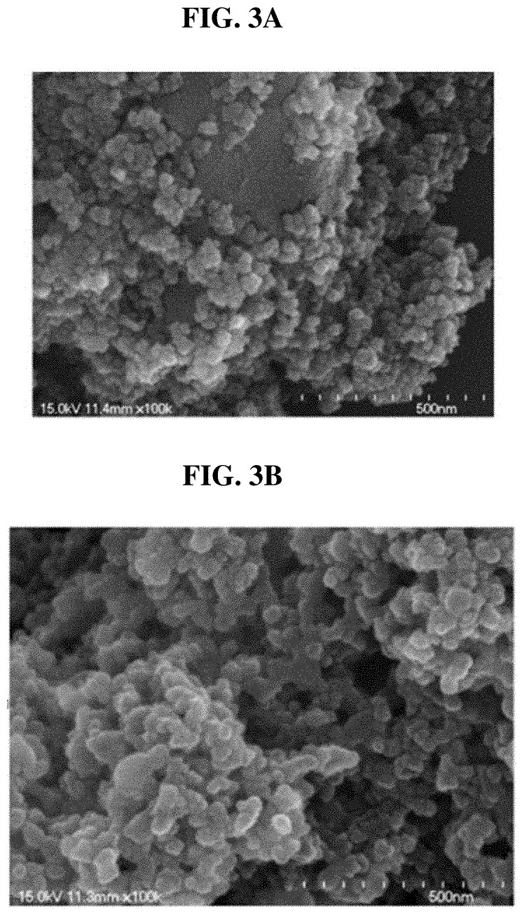

FIG. 3 is a set of SEM images of pure Fe₃O₄ magnetic nanoparticles (FIG. 3A) and a structure LF-bis-MPA-MNP (FIG. 3B) prepared in the Synthesis Example of the Example according to an exemplary embodiment of the present invention. A uniform particle size distribution and shape can be confirmed from the SEM image.

(2) Size Distribution Plot

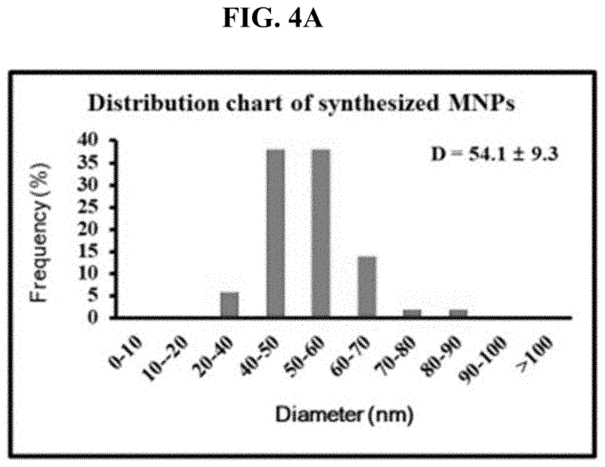

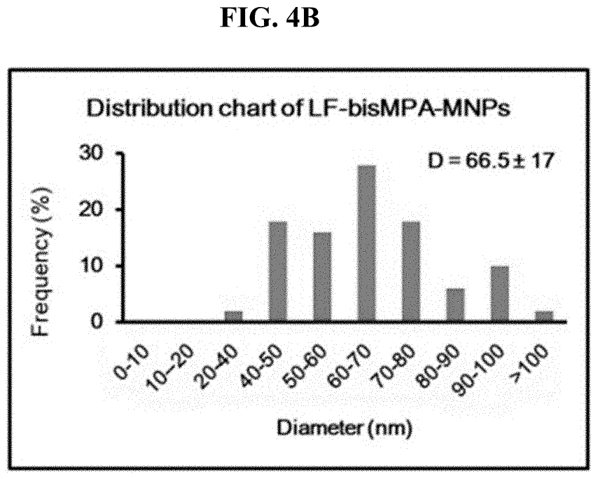

FIG. 4 illustrates, as a graph, a size distribution plot analyzed by ImageJ by randomly measuring the sizes of pure Fe₃O₄ magnetic nanoparticles (FIG. 4A) and 200 particles of a structure LF-bis-MPA-MNP (FIG. 4B) prepared in the Synthesis Example of the Example according to an exemplary embodiment of the present invention. The average sizes of the pure magnetic nanoparticles and the structure (LF-bisMPA-MNP) prepared in the Synthesis Example were approximately 54.1±9.1 and 66.5±17 nm, respectively. Looking at the graph, it can be seen that the particle size distribution is uniform.

(3) Zeta Potential

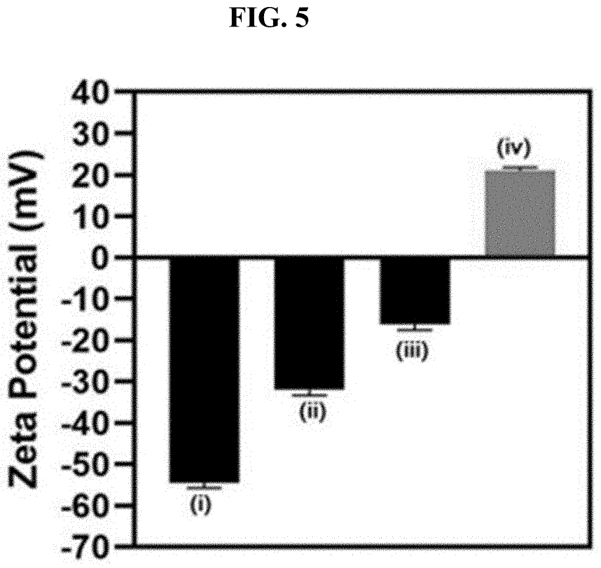

FIG. 5 illustrates the zeta potentials of commercially available Fe₃O₄ magnetic nanoparticles, synthesized Fe₃O₄ magnetic nanoparticles, bisMPA-MNP, and LF-bis-MPA-MNP according to the present invention. Before the surface of LF-bis-MPA-MNP was coated with lactoferrin (LF), commercially available Fe₃O₄ magnetic nanoparticles, synthesized Fe₃O₄ magnetic nanoparticles, and bis-MPA-MNP showed a negative charge of −54.5±1.27, −32.03±1.31 and −16.2±1.45 mV, respectively. A negative zeta potential of bis-MPA-MNP dispersed in distilled water indicates that there are carboxylic groups on the surface of bis-MPA-MNP. The zeta potential changed after the conjugation of lactoferrin (LF), exhibiting a positive charge of 20.97±0.83 mV. This means that lactoferrin having a positive surface charge was successfully conjugated to the surface of the magnetic nanoparticles.

(4) FT-IR Spectrum

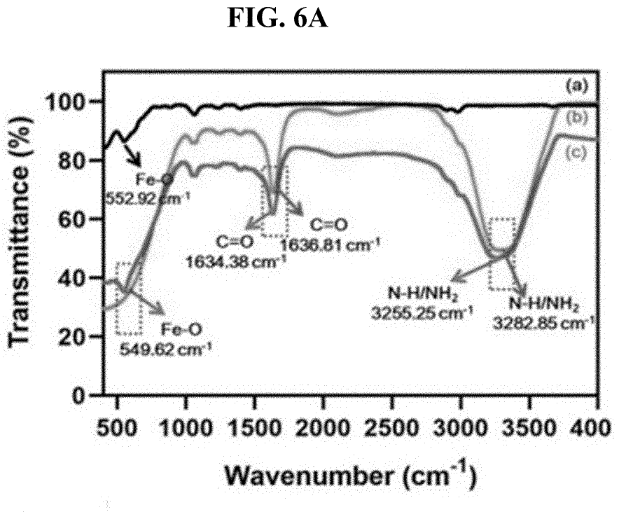

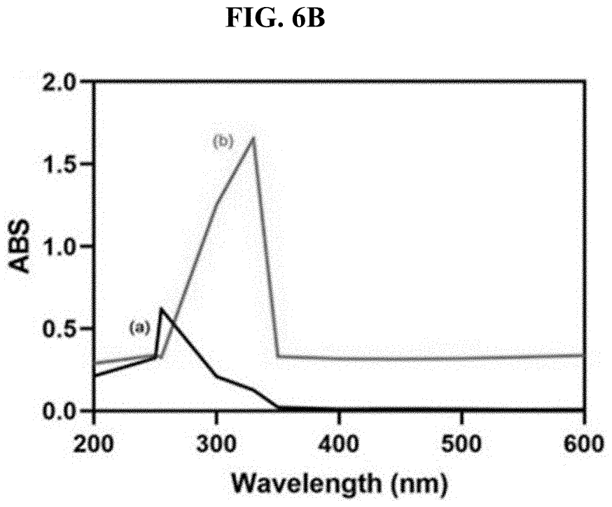

FIG. 6A is a view illustrating FT-IR spectra. Line (a) is for the synthesized MNP core, Line (b) is for LF, and Line (c) is for LF-bis-MPA-MNP. The characteristic peak on Line (c), for example, the characteristic peak at 3255.25 cm⁻¹, represents a NH₂ peak due to the successful conjugation of LF. FIG. 6A means that LF was completely attached to Bis-MPA-MNP.

(5) UV-Visible Light Spectrum

In FIG. 6B, it was confirmed whether or not lactoferrin (LF) was attached to MNPs using a UV-visible light spectrometer. Line (a) is for the MNP and Line (b) is for LF-bis-MPA-MNP. The characteristic absorption peak of LF-bis-MPA-MNP of 330 nm appeared on Line (b). This confirmed that LF was well attached.

### [Evaluation Example 2] Confirmation of Pathogen Concentration Efficiency

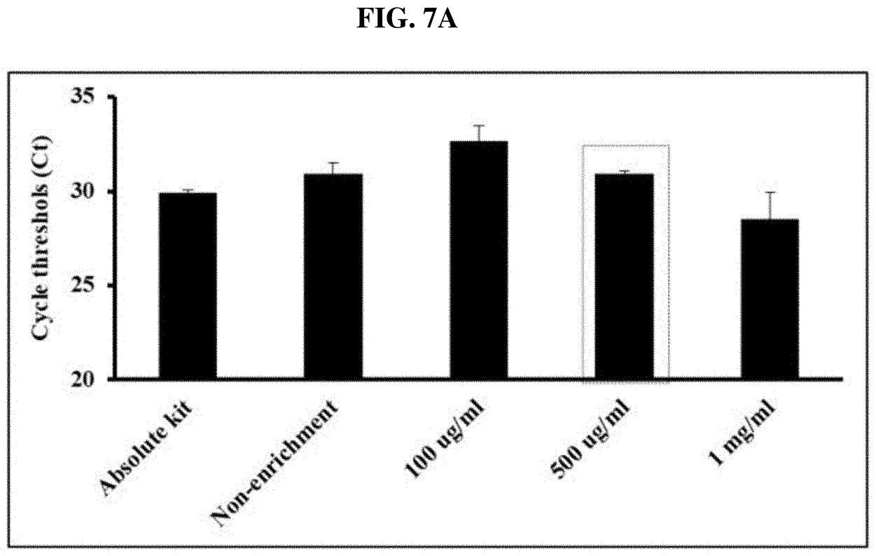

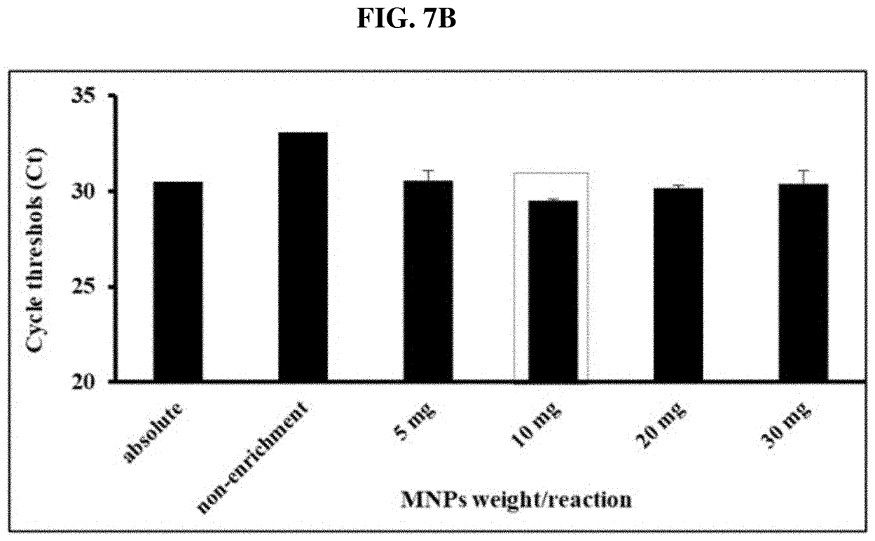

First, the conditions for concentrating a pathogen or isolating extracellular vesicles using LF-bis-MPA-MNP were optimized, and the results are illustrated in FIGS. 7A and 7B. FIG. 7A is LF concentration optimization for coating onto MNPs, and FIG. 7B illustrates the experimental results for optimizing the concentration of LF-bis-MPA-MNP.

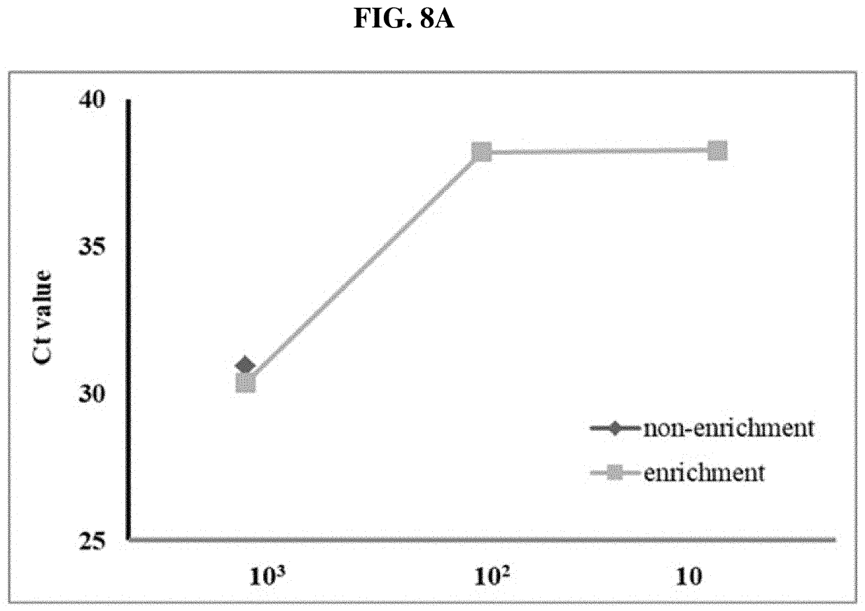

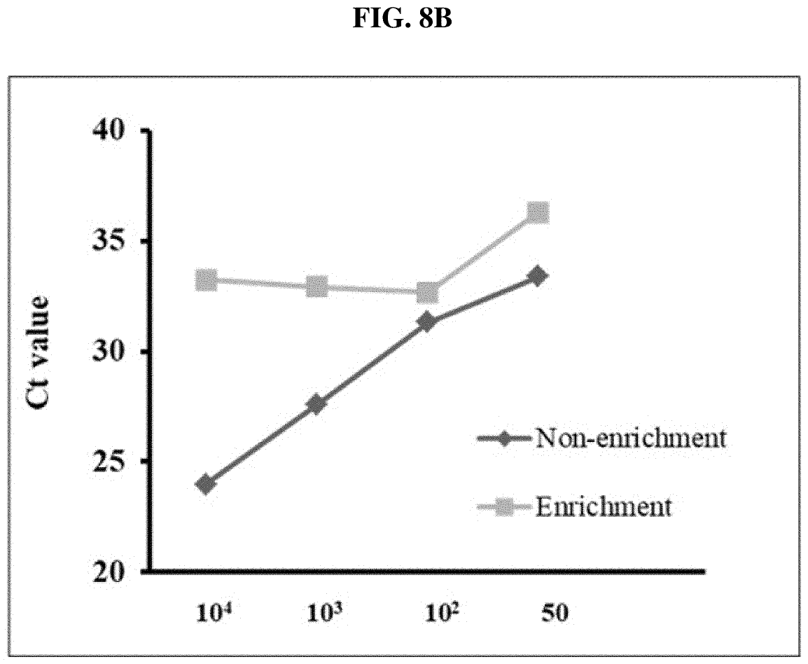

The results of concentrating and detecting Salmonella (Gram-negative bacterium) (FIG. 8A) and Brucella (FIG. 8B) in PBS samples using the structure of the present invention at various dilutions are illustrated in FIGS. 8A and 8B. From FIGS. 8A and 8B, it could be confirmed that various pathogens can be concentrated and detected using the structure according to the present invention.

### [Evaluation Example 3] Comparison of Exosome Isolation Methods

Extracellular vesicles were isolated using ultracentrifugation (UC), a total exosome isolation (TEI) kit, a DTS chip, and LF-bis-MPA-MNP.

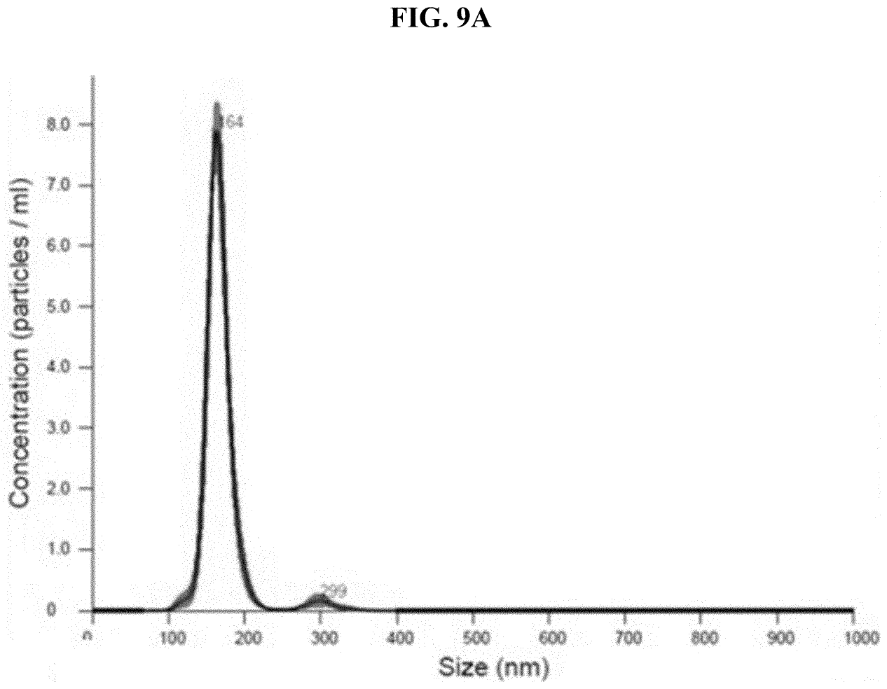

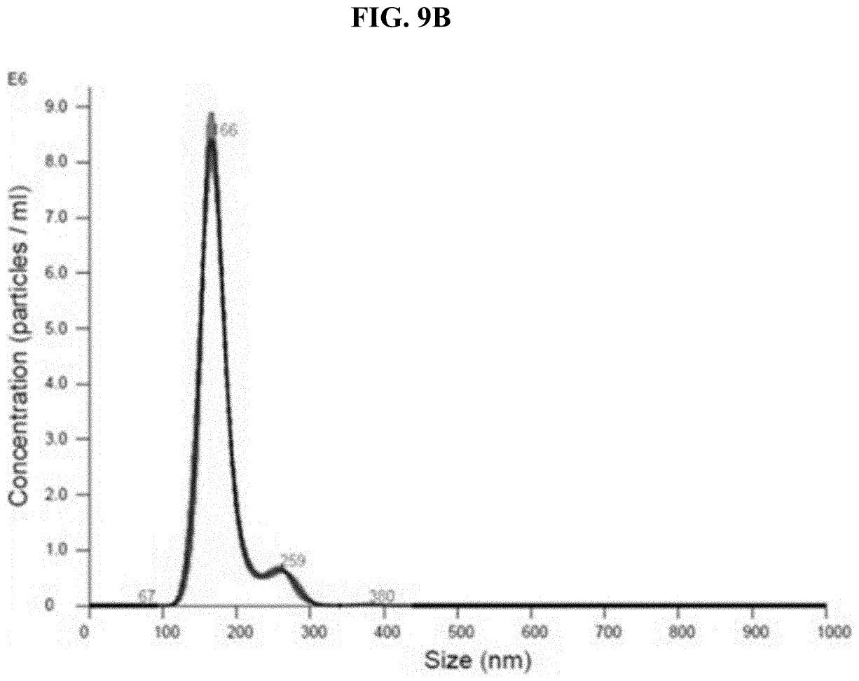

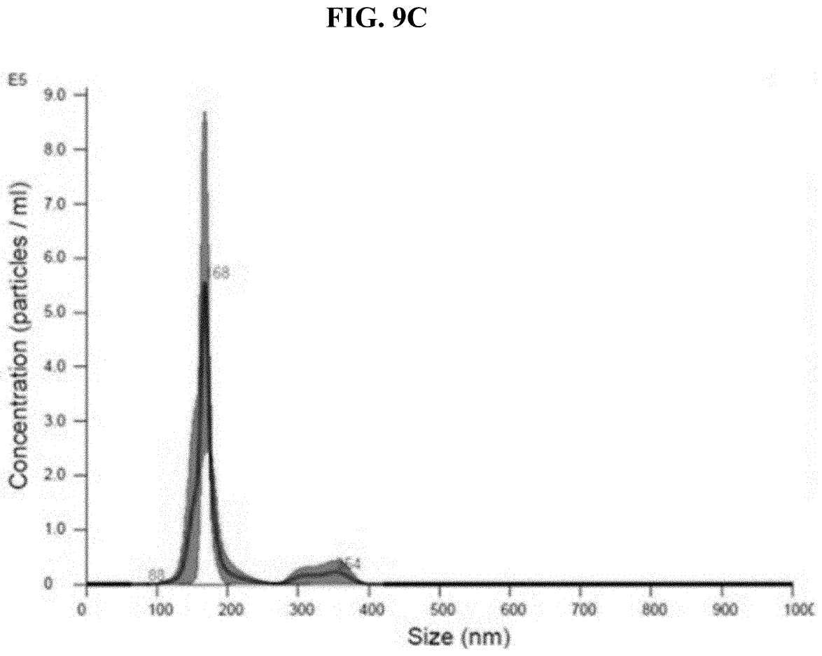

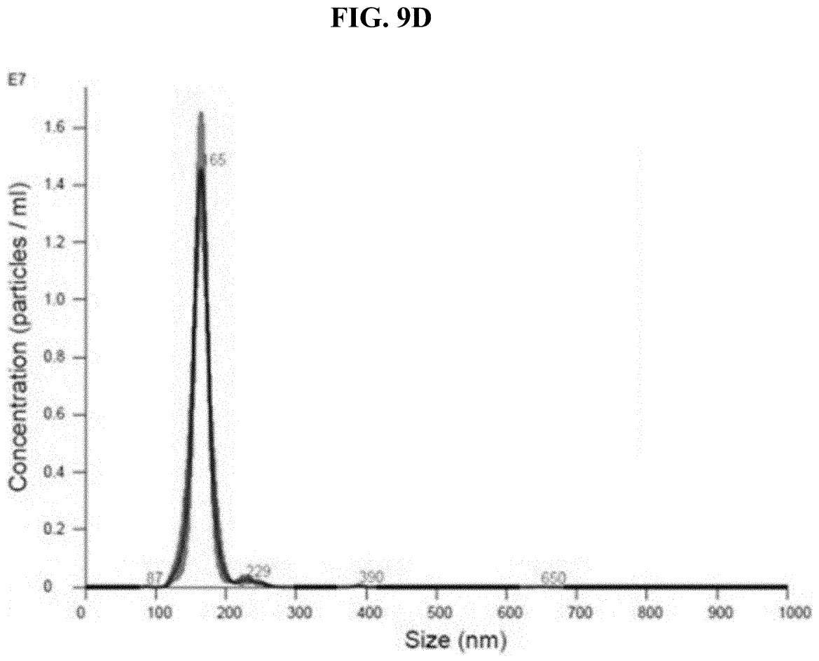

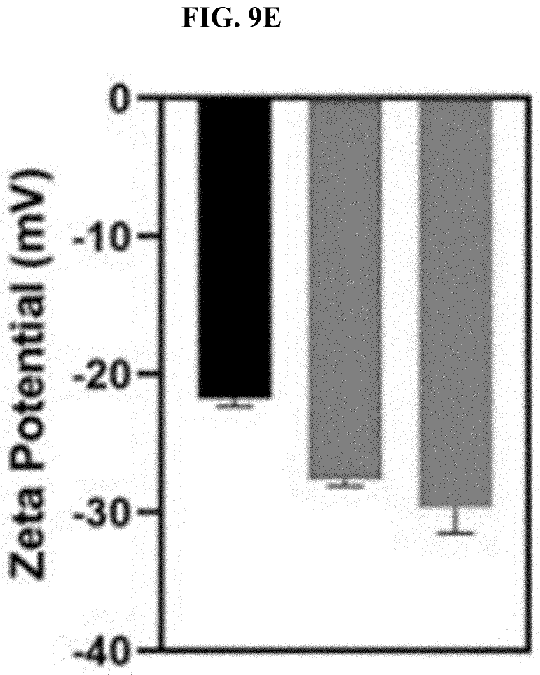

From FIGS. 9A to 9D, it can be seen that there is no difference in size or shape between extracellular vesicles (EVs) isolated by UC and LF-bis-MPA-MNP. The average diameters of the particles shown on the graph are equivalent to the sizes of EVs isolated by UC, TEI, the DTS chip and LF-bis-MPA-MNP, and were 163.1±27.7, 165.9±32.5, 167.6±56.6, and 164.5±21.9 nm, respectively.

The charge density distribution around the particles causes a difference in electrostatic potential. Zeta potentials were measured to examine EV stability and integrity, and the results are illustrated in FIG. 9E. The zeta potentials of the isolated EVs when using UC, TEI, and LF-bis-MPA-MNP were −21.80±0.51 mV, −27.61±0.46 mV, and −29.64±1.88 mV, respectively. There was no significant difference between the isolation methods, and due to the plasma membrane structure of EVs, they exhibited a negative surface charge in a range of −21.8 to −29.64 mV. This means that EV stability in a solution is excellent.

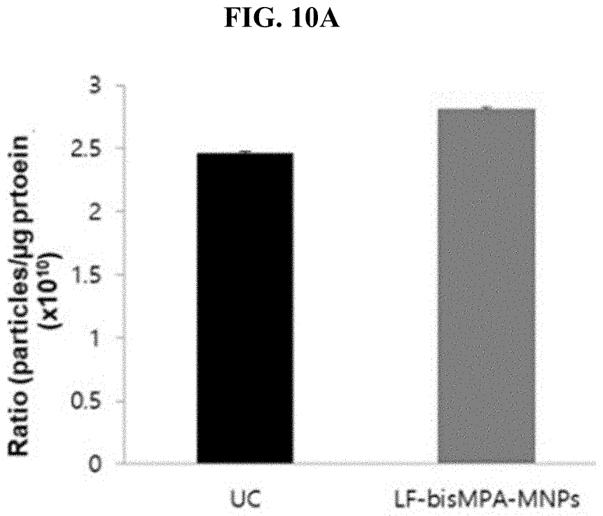

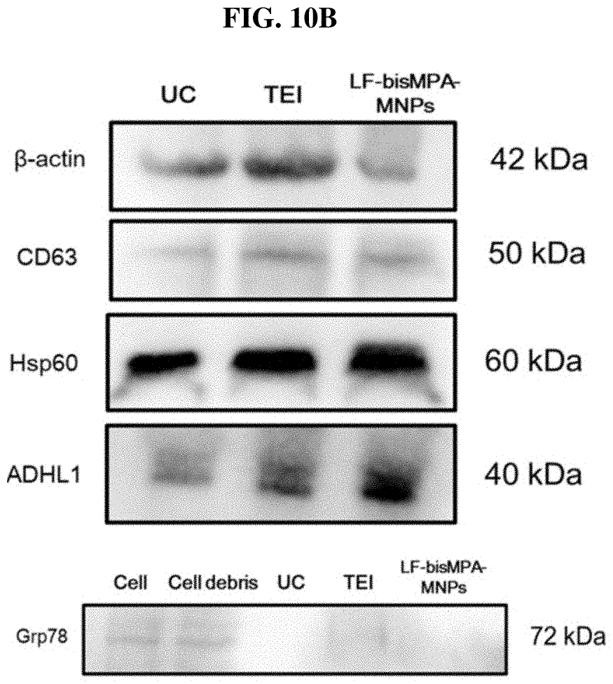

Exosomes display a specific protein, for example, a specific protein such as CD9, CD64, or CD81, on their surface. Therefore, the purity thereof was checked by immunoblotting. The results thereof are illustrated in FIGS. 10A and 10B. CD63 was found in EVs isolated by UC, TEI, and LF-bis-MPA-MNP (EV from HCT116 rectal cancer cells), but not in cell suspensions. It can be seen that small EVs were also successfully isolated by exosome marker detection (FIG. 10A). The detection intensities for the CD63 marker were different, and the signal was the weakest in the UC method. Next, ARF-6 was detected in cell debris. Grp78 (apoptotic body) was found in cell suspensions and cell debris, but not in samples isolated by UC, TEI, and LF-bis-MPA-MNP. This means that no apoptotic body was included in the isolated EVs. EV isolation by three protocols was evaluated using specific antibodies against Hsp60 (from the CCM of rectal cancer HCT116) and ADHL1 (from the CCM of HepG2 liver cancer cells). Hsp60 and ADHL1 were found in EVs isolated by the three methods, but were expressed slightly higher in EVs isolated by LF-bis-MPA-MNP.

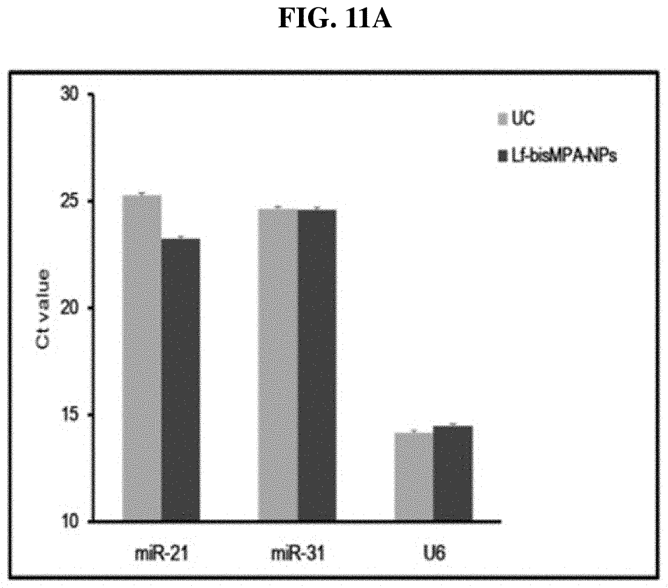

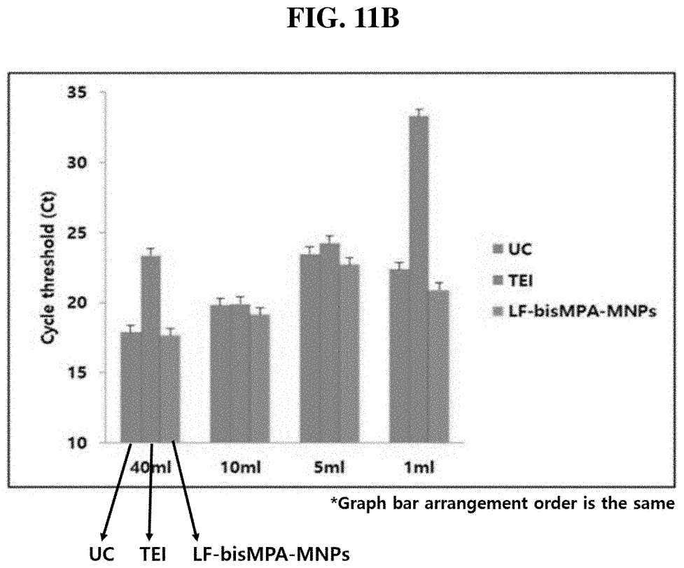

Exosomal miRNAs were extracted from exosomes derived from the CCM of rectal cancer HCT116 cells. EV isolation efficiencies by UC and LF-bis-MPA-MNP were compared through Ct values after cDNA synthesis and qRT-PCR on targets miR-21, miR-31 and U6. The results thereof are illustrated in FIGS. 11A and 11B. FIG. 11A shows that miR-21 is more efficiently recovered from exosomes isolated by LF-bisMPA-MNP than by UC. However, in the case of Ct values for miR-31 and U6, there was no significant difference in Ct values between UC and LF-bis-MPA-MNP. Such results mean that LF-bis-MPA-MNP may be used as a suitable method for EV isolation and is comparable to UC or TEI.

Such a series of results described above mean that the LF-bis-MPA-MNP according to the present invention is a simple and novel method capable of isolating EVs with higher efficiency and purity than UC, TEI, and the DTS chip. Furthermore, ultracentrifugation or complicated and slow equipment need not to be used. Accordingly, a simple and quick method using magnetism may be provided.

Although the present invention has been described above with reference to preferred exemplary embodiments of the present invention, a person with ordinary skill in the art can understand that the present invention can be modified and changed in various ways in a range not departing from the spirit and scope of the present invention described in the following claims.

What is claimed is:

1. A nanostructure for isolating or concentrating extracellular vesicles or a pathogen, comprising a transferring family protein linked on magnetic nanoparticles.

2. The nanostructure of claim 1, wherein the magnetic nanoparticles are iron oxide (Fe₃O₄) nanoparticles.

3. The nanostructure of claim 1, wherein the magnetic nanoparticles have a diameter of 10 to 120 nm.

4. The nanostructure of claim 1, wherein the nanostructure has a size of 20 to 150 nm.

5. The nanostructure of claim 1, wherein the nanostructure exhibits a positive surface charge.

6. The nanostructure of claim 1, wherein the transferrin family protein is at least one selected from the group consisting of lactoferrin, serotransferrin, ovotransferrin, milk transferrin, and melanotransferrin.

7. The nanostructure of claim 1, further comprising a polyfunctional crosslinking portion and/or a linking portion derived from a silane coupling agent on the magnetic nanoparticles.

8. The nanostructure of claim 7, wherein the polyfunctional crosslinking portion is derived from a carboxylic acid having an alcohol-based functional portion.

9. The nanostructure of claim 7, wherein the nanostructure comprises a polyfunctional crosslinking portion and has the shape of a dendrimer.

10. The nanostructure of claim 1, wherein the silane coupling agent is an amino-based silane coupling agent selected from 3-aminopropyltriethoxysilane (APTES) and 3-aminopropyltrimethoxysilane (APTMS).

11. The nanostructure of claim 1, wherein the nanostructure has the following Chemical Formula 1: 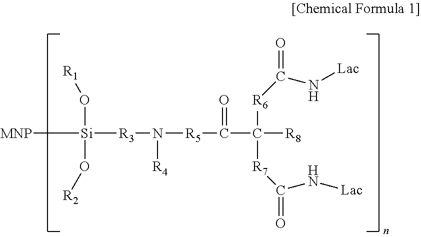
in Chemical Formula 1,
MNP represents a magnetic nanoparticle,
Lac represents a transferrin family protein,
R₁ and R₂ are each independently an alkyl group having 1 to 5 carbon atoms,
R₃, R₅, R₆, and R₇ are each independently an alkylene group having 1 to 5 carbon atoms,
R₄ is a hydrogen atom or an alkyl group having 1 to 5 carbon atoms, and
n is an integer greater than or equal to 1.

12. A method for isolating or concentrating extracellular vesicles or a pathogen using the nanostructure of claim 1.

13. The method of claim 12, wherein the method comprises (a) a step of bringing the nanostructure of claim 1 into contact with a pathogen or extracellular vesicles and (b) a step of applying a magnetic field.

14. The method of claim 13, further comprising: (c) a step of isolating a material captured by the magnetic force from the magnetic field and (d) a step of isolating the pathogen or extracellular vesicles from the structure.

15. The method of claim 12, wherein the pathogen is a virus, bacterium or fungus capable of binding to a transferrin family protein.

16. The method of claim 12, wherein the extracellular vesicles comprise exosomes.

17. A method for preparing a nanostructure for isolating or concentrating extracellular vesicles or a pathogen, the method comprising: (i) a step of treating magnetic nanoparticles with a silane coupling agent;
(ii) a step of treating the product of Step (i) with a polyfunctional crosslinking agent;
(iii) a step of converting a polyfunctional end group of the product of Step (ii) to a thiol group;
(iv) a step of treating the product of Step (iii) with a maleimide-based crosslinking agent; and
(v) a step of treating the product of Step (iv) with a transferrin family protein.
